(19) United States
(12) Patent Application Publication
(10) Pub. No.: US 2025/0292511 A1
(43) Pub. Date: Sep. 18, 2025

(54) MACHINE LEARNING FOR THREE-DIMENSIONAL MESH GENERATION BASED ON IMAGES

(71) Applicant: ResMed Pty Ltd., Sydney (AU)

(72) Inventors: Michael Christopher HOGG, Sydney (AU); Hongjiang YU, Sydney (AU); Paul Andrew DICKENS, Sydney (AU); Liam HOLLEY, Sydney (AU); Kristy Tzu Ying TAN, Sydney (AU); Andrea Kaye GRIGSBY, London (GB); Stephanie KLINKENBERG, Sydney (AU)

(73) Assignee: ResMed Corp., San Diego, CA (US)

(21) Appl. No.: 19/175,848

(22) Filed: Apr. 10, 2025

Related U.S. Application Data
(63) Continuation of application No. 19/072,009, filed on Mar. 6, 2025.
(60) Provisional application No. 63/565,443, filed on Mar. 14, 2024.
(60) Provisional application No. 63/671,994, filed on Jul. 16, 2024.

(51) Int. Cl. G06T 17/20; A61M 16/06

(52) U.S. Cl. CPC G06T 17/205; A61M 16/0605; A61M 16/0683; A61M 2016/0661; G06T 2210/41

(57) ABSTRACT

Techniques for improved machine learning are provided. A set of two-dimensional images of a user is accessed. A three-dimensional mesh depicting a head of the user is generated based on processing the set of two-dimensional images using a machine learning model, where the three-dimensional mesh is scaled to a size of the head of the user. The three-dimensional mesh is modified to remove one or more facial expressions. A set of facial measurements is determined based on the modified three-dimensional mesh, and a user interface is selected for the user based on the set of facial measurements.

### CROSS-REFERENCE TO RELATED APPLICATIONS

This application claims priority to U.S. Provisional Application No. 63/671,994, filed Jul. 16, 2024, the entire contents of which is incorporated herein by reference in their entirety. In addition, this application claims priority to U.S. Non-Provisional application Ser. No. 19/072,009, filed Mar. 6, 2025, which claims the benefit of U.S. Provisional Application No. 63/565,443, filed Mar. 14, 2024, the entire contents of each of which is incorporated herein by reference in their entirety.

### INTRODUCTION

Embodiments of the present disclosure generally relate to computer vision and machine learning. More specifically, embodiments relate to using machine learning to generate three-dimensional meshes based on image data.

In a wide variety of medical (and non-medical) settings, accurate facial or head measurements are relied upon to drive decisions and selections for the user. For example, in many cases, the particular dimensions of the face of the individual user are needed to help design, construct, and/or select an appropriate mask that will fit the user's face comfortably and completely. As one example, continuous positive airway pressure (CPAP) machines generally use a mask or nosepiece to deliver constant and steady air pressure to users during sleep. However, for the system to operate properly (as well as to improve the user experience and health), it is important that the mask fit properly (e.g., comfortably, and without leaks around the face).

In some conventional systems, users can visit a physical environment (e.g., the office of a healthcare provider or mask distributor) to try on various masks. However, this requires physical presence of the user, which is not always possible due to factors such as location or remoteness of the user and/or provider, ability to travel, available time for the user, and the like. Further, the user is limited to the available (preconfigured) mask sizes and dimensions, and generally must manually try a number of them to find a correct fit, which can present problems when sterility is required.

In some conventional systems, attempts have been made to measure or estimate facial dimensions of the user, in order to drive mask selection or design. For example, some approaches involve the user measuring their own face, such as by using a ruler, a coin or other object with a known size, and the like. However, such approaches have proven to be inaccurate and frustrating to the user leading to poor outcomes. For example, obtaining the wrong mask based on inaccurate measurements may lead to a mask that does not work well for its intended purpose, which can in-turn affect a user's condition, treatment, and outcomes.

Additionally, some approaches have attempted to improve on manual measurements by using specialized devices or systems, such as cameras configured to capture three-dimensional data including depth. These specialized devices are complex, expensive, and frequently unavailable. Further, they typically still require the user to physically travel to the location of the device (or require the device to be physically brought to the user), significantly limiting their use.

Improved systems and techniques to determine facial measurements are needed.

### SUMMARY

According to one embodiment presented in this disclosure, a method is provided. The method includes: accessing a set of two-dimensional images of a user; generating, based on processing the set of two-dimensional images using a first machine learning model, a three-dimensional mesh depicting a head of the user, wherein the three-dimensional mesh is scaled to a size of the head of the user; modifying the three-dimensional mesh to remove one or more facial expressions; determining a set of facial measurements based on the modified three-dimensional mesh; and selecting a user interface for the user based on the set of facial measurements.

Other embodiments provide processing systems configured to perform the aforementioned methods as well as those described herein; non-transitory, computer-readable media comprising instructions that, when executed by one or more processors of a processing system, cause the processing system to perform the aforementioned methods as well as those described herein; a computer program product embodied on a computer readable storage medium comprising code for performing the aforementioned methods as well as those further described herein; and a processing system comprising means for performing the aforementioned methods as well as those further described herein.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 depicts an example workflow for generating meshes and selecting user interfaces, according to some embodiments of the present disclosure.

FIG. 2 depicts an example workflow to facilitate image data collection for improved mesh generation, according to some embodiments of the present disclosure.

FIG. 3 depicts an example workflow for improved interface selection based on generated meshes, according to some embodiments of the present disclosure.

FIGS. 4A, 4B, and 4C depict an example breathing exercise for improved user interface selection, according to some embodiments of the present disclosure.

FIG. 5 is a flow diagram depicting a method for using machine learning model(s) to generate three-dimensional meshes and select interface components, according to some embodiments of the present disclosure.

FIG. 6 is a flow diagram depicting a method for collecting and evaluating image data, according to some embodiments of the present disclosure.

FIG. 7 is a flow diagram depicting a method for improved interface selection, according to some embodiments of the present disclosure.

FIG. 8 is a flow diagram depicting a method for using machine learning to select user interfaces, according to some embodiments of the present disclosure.

FIG. 9 depicts an example computing device configured to perform various embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present disclosure provide apparatuses, methods, processing systems, and computer-readable mediums for machine learning (ML)-based interface selections based on three-dimensional meshes.

In some embodiments, a measurement system is provided. The measurement system may be configured to evaluate a set of images captured via an imaging sensor (e.g., a webcam) using machine learning to generate a three-dimensional mesh corresponding to the face of a user depicted in the image(s). In some embodiments, such meshes may be used to generate accurate biometric measurements. Beneficially, the system can use two-dimensional images to generate the three-dimensional mesh, and need not rely on complex three-dimensional imaging systems. This enables the system to be implemented using a wide range of widely available imaging sensors, including web cameras, mobile device cameras, and the like. In some embodiments, a deep learning model is trained end-to-end to generate three-dimensional meshes having appropriate size or scale, relative to the user's face. That is, the mesh may be scaled according to the size of the user's face, ensuring that measurements taken based on the mesh (e.g., nose size, mouth position, and the like) are accurate.

In some embodiments, the user may directed to move a body part, such as their face, through a range of motion while an imaging sensor captures images of the body part at different angles. Trained machine learning models may then be used to evaluate the image(s) to ensure they are satisfactory (e.g., to ensure the user's ear is visible in one or more pictures) to ensure accurate mesh generation.

In some embodiments, the measurement system may perform various operations using the generated mesh. In some embodiments, the measurement system may morph or deform the mesh to remove facial expressions, if any, depicted in the image. For example, if the user is smiling, raising their eyebrows, or making some other expression, the system may modify the mesh to remove such expression(s). A wide variety of facial expressions may result in inaccurate facial measurements (e.g., inaccurate nostril size prediction due to deformation of the user's skin when they smile). Therefore, by manipulating the three-dimensional mesh to remove such expressions, the measurement system can ensure that the captured measurement data is highly accurate.

In some embodiments, once the three-dimensional facial mesh has been generated and processed appropriately (e.g., to remove facial expressions), the mesh can be used to estimate, calculate, compute, or otherwise determine a set of facial measurements of the user. The particular measurements collected may vary depending on the particular implementation and task. For example, in some embodiments, the measurement system generates the facial measurements to facilitate selecting and/or fitting of one or more devices or components designed to be worn on the head or face of the user, such as user interfaces (e.g., masks) for respiratory therapy (e.g., CPAP).

For example, in some embodiments, the measurement system may determine measurements such as the face height, nose width, nose depth, nostril size, and the like. In various embodiments, the particular facial measurements that are determined and used may vary depending on the particular task (e.g., to allow determination of proper sizing for conduits (e.g., for tube-up masks), head gear, nostril sizes for pillow masks, and the like). As discussed below in more detail, these measurements can be used to select, design, customize, or otherwise retrieve a facial device or user interface for the user, such as an appropriately-fitted mask for the user, to ensure functionality, comfort, and stability.

### Example Workflow for Generating Meshes and Selecting User Interfaces

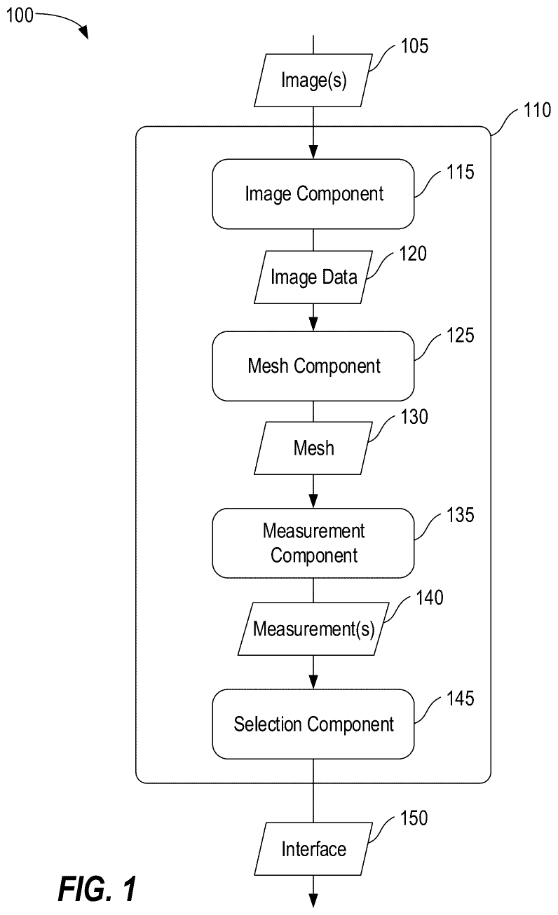

FIG. 1 depicts an example workflow 100 for generating meshes and selecting user interfaces, according to some embodiments of the present disclosure.

In the illustrated example, a measurement system 110 accesses a set of image(s) 105 and generates or selects an interface 150 (e.g. a recommendation or selection of the interface 150) based on the image(s) 105. As used herein, “accessing” data may generally include receiving, retrieving, requesting, obtaining, collecting, capturing, measuring, or otherwise gaining access to the data. For example, in some embodiments, the image(s) 105 may be captured via one or more imaging sensors (e.g., a webcam or a camera on a smartphone) and may be transmitted to the measurement system 110 via one or more communication links. In some embodiments, the measurement system 110 is implemented as a cloud-based service that evaluates user images 105 to generate the interfaces 150. For example, users may use an application to capture the image(s) 105 on their local devices (e.g., the user's laptop or phone), and may then upload the image(s) 105 to the measurement system 110 for evaluation. Generally, the measurement system 110 may be implemented using hardware, software, or a combination of hardware and software. Further, though illustrated as a discrete system for conceptual clarity, in some embodiments, the operations of the measurement system 110 may be combined or distributed across any number and variety of devices and systems.

In some embodiments, the image(s) 105 generally correspond to two-dimensional images that depict the head and/or face of the user. In some embodiments, the image(s) 105 depict the user from multiple angles or orientations. For example, the image(s) 105 may include a frontal image (e.g., captured while the user's face is angled directly towards the imaging sensor, such that the image depicts the face of the user from straight on), one or more side or profile images (e.g., captured while the user turned their face towards the left and/or right side of the imaging sensor, such that the image(s) depict the side of the user's face and/or the user's ear(s)), a bottom image (e.g., captured while the user looked upwards relative to the imaging sensor, such that the image depicts the user's chin, neck, and/or nostrils), and/or a top image (e.g., captured while the user looked downwards relative to the imaging sensor, such that the image depicts the top of the user's head).

Although not depicted in the illustrated example, in some aspects, the measurement system 110 may receive various other data, such as metadata associated with one or more images 105 (e.g., indicating characteristics such as the field of view (FOV) or focal length of the camera that captured the image(s) 105).

In the illustrated workflow 100, the image(s) 105 are accessed by an image component 115. The image component 115 generally facilitates collection and evaluation of the images 105. The particular operations performed by the image component 115 may vary depending on the particular implementation. For example, in some embodiments, the image component 115 may perform various preprocessing operations, such as to enhance contrast, reduce noise, resize the images, crop the images, perform color correction on the images, perform feature extraction, and the like. In some embodiments, the image component 115 may evaluate one or more of the image(s) 105 to confirm that the image(s) 105 are suitable for mesh generation. For example, the image component 115 may use one or more machine learning models to detect the presence (or absence) of various facial features or landmarks that are useful in mesh generation, such as the car(s) of the user in the profile image(s). In some embodiments, if such landmarks are not visible, the image component 115 may request additional image(s) 105 to improve the measurement process.

As illustrated, the image component 115 provides image data 120 to a mesh component 125. The image data 120 may correspond to or comprise the image(s) 105 themselves, and/or may correspond to the image(s) 105 after preprocessing operation(s) are applied, such as noise reduction or resizing. In some embodiments, the image data 120 corresponds to or comprises the results of feature extraction. That is, the image data 120 may comprise feature map(s) generated for the image(s) 105.

The mesh component 125 processes the image data 120 to generate a mesh 130. In some embodiments, the mesh component 125 uses one or more machine learning models to generate the mesh 130. For example, the mesh component 125 may use a deep learning model (e.g., a convolutional neural network) to generate the mesh 130. In some embodiments, the mesh component 125 uses the machine learning model(s) to fit a statistical shape model representing a statistically average face to the image data 120, causing the mesh 130 to depict or correspond to the face of the user.

In some embodiments, the mesh component 125 uses a camera model to scale the mesh 130. For example, in some embodiments, the mesh component 125 may determine the FOV of the camera used to capture the image(s) 105 (e.g., from metadata associated with the images 105, and/or by processing the images themselves). In some embodiments, the perceived size of various facial landmarks may change as the landmarks move closer to or further from the camera. Based on the perceived changes in size of the landmark(s) (e.g., the user's head, or more granular landmarks such as eyes or ears), in some embodiments, the mesh component 125 can use a camera model to determine or infer the FOV of the camera and/or the distance between the camera and the landmark(s). Using this information, in some embodiments, the mesh component 125 can determine the scale of the face or features therein. For example, after determining that the user's nose is N millimeters away from the camera and that the FOV of the camera is X degrees, the mesh component 125 may determine the actual size of the user's nose (e.g., in millimeters).

In some embodiments, rather than using a camera model to generate or scale the mesh, the mesh component 125 may use a deep learning model that generates an appropriately scaled mesh 130. For example, the model may be trained based on facial exemplars to generate the mesh 130 in a way that inherently understands the scale of the face, without using a separate camera model (e.g., without explicitly determining or evaluating the FOV of the camera, for example). In some embodiments, to train the model, the measurement system 110 (or another system) may use relatively dense exemplars, such as images and corresponding meshes or dense coordinates of facial landmarks.

In some embodiments, some or all of the training data comprises synthetic data. For example, accurate three-dimensional meshes or models of synthetic heads and/or faces may be generated using various computer programs (e.g., models of people that are not real individuals or users, but where the models are nevertheless realistic). In some embodiments, the measurement system 110 (or another system) may render image(s) depicting the modeled head from various angles (e.g., by placing a virtual camera in the virtual space at various positions around the head). These images may be used as the training input to the model, paired with some or all of the mesh itself (e.g., data points in three-dimensional space, such as defining various landmarks of the face) used as the target output. In some embodiments, in addition to or instead of using synthetic data to train the model, the training data may include real data (e.g., real images of a user, coupled with highly accurate three-dimensional data points). For example, users may volunteer to use a scanning device capable of capturing image data and three-dimensional positioning data for their face.

However, such scanning devices are often cumbersome, expensive, and difficult to use. Further, scanning actual faces of users to train the model may implicate various privacy concerns. Experimentation has shown that using purely synthetic data to train the model can nevertheless provide robust mesh generation during runtime.

In the illustrated workflow 100, the mesh 130 is a three-dimensional mesh representing at least a portion of the user's head and face. For example, the mesh 130 may depict the user's face, a portion of their neck, and/or a portion of their head (e.g., including the cars). The mesh 130 is accessed by a measurement component 135. In the illustrated example, the measurement component 135 generates a set of measurements 140 based on the mesh 130.

In some embodiments, prior to generating the measurements 140, the measurement component 135 may apply one or more preprocessing operations. For example, the measurement component 135 may morph or deform the mesh 130 to remove the facial expression(s) of the user, if any, resulting in a mesh that reflects the face of the user in a neutral expression.

In embodiments, as discussed above, the particular measurements captured by the measurement component 135 may vary depending on the particular implementation and task. For example, in some aspects, the measurement component 135 may evaluate the mesh 130 to determine features such as the nose width, nose height, and/or nose depth of the user. Such measurements may be useful to select or provide facial devices such as a face mask (e.g., a respiratory therapy mask) that covers the nose of the user. As another example, in some embodiments, the measurement component 135 may measure the height and/or width of the user's mouth, and/or the positioning of the mouth relative to the nose, for similar reasons.

In some embodiments, the measurement component 135 may determine the overall size of the user's head (e.g., the circumference of the user's head), which may be a useful metric for conduit and/or headgear sizing (e.g., to select a conduit that is sufficiently large to comfortably reach around the user's head without being too large such that it is uncomfortable, and/or to select headgear that will fit comfortably). As used herein, “headgear” refers to the straps, bands, or other components used to secure the user interface to the user's nose, mouth, or both. As used herein, the “conduit” refers to a tube that connects the user interface (e.g., a CPAP mask) to the respiratory therapy device (e.g., the flow generator) and provides airflow to the user, from the flow generator, via the interface. In some aspects, to facilitate conduit sizing, the measurement component 135 may determine the length of the conduit path along the user's face (e.g., along the path where the conduit is designed to sit, such as from the nose and/or mouth and up over each car).

In some embodiments, the measurement component 135 may determine the nostril size of the user based on the mesh 130. For example, the measurement component 135 may characterize the nostril(s) of the user using four parameters defining an ellipse: the major axis and minor axis of the ellipse, the rotation of the nostril/ellipse relative to a fixed orientation (e.g., relative to the plane of the face), and the distance between the nostril/ellipse and the centerline of the mesh 130 (e.g., the centerline of the user's face). Although some examples discussed use an ellipse to define the nostril measurements, in some embodiments, the measurement component 135 may use a variety of polygons having any number of sides to define the shape of the nostril.

In the illustrated example, the measurements 140 (also referred to in some embodiments as facial measurements) are accessed by a selection component 145. The selection component 145 evaluates the measurements 140 to select or generate the interface 150. In some embodiments, the selection component 145 may evaluate one or more of the measurements 140 using one or more thresholds or mappings to select various components of the interface 150. For example, based on the nose size and/or shape of the user, the selection component 145 may evaluate predefined mappings indicating which interface(s) will fit best or be most comfortable. As one example, the selection component 145 may determine that a first nasal-only mask may be too small to comfortably fit, that a second nasal-only mask will be too large (e.g., such that air leak occurs), and/or that a third nasal-only mask will fit well and be comfortable. As another example, based on the measurements 140, the selection component 145 may determine that the user should use a particular type or model of full-face masks (e.g., an oronasal mask).

In some embodiments, the selection component 145 may evaluate some or all of the measurements 140 to select a nasal pillow size for the user. Nasal pillows are generally soft inserts that fit partially into the nostrils of the user, providing airflow via the nostrils (whereas a nasal mask fits over the nose, and a full-face mask fits over the nose and mouth). In some embodiments, the selection component 145 uses a classifier machine learning model to select the pillow size based on the nostril measurements. For example, the classifier may process the measurements such as nostril major and minor axes, rotation, and/or distance to centerline to generate a classification indicating which size pillow would fit the user best. In some embodiments, the classifier may be a relatively small or simple machine learning model. The measurement system 110 (or another system) may train the nostril classifier using labeled exemplars. For example, the training data may include nostril measurements (as discussed above) of one or more users, where the label for each training sample indicates the pillow size that the user found most comfortable (or that otherwise led to the best results, such as the minimum air leakage).

Generally, the generated or selected interface 150 may include selections for a variety of interface components, as discussed above. For example, the interface 150 may indicate a recommended user interface style or design (e.g., nasal only, full-face, or nasal pillow), a recommended model or size of interface (e.g., from a set of alternative options), a recommended conduit sizing, a recommended headgear sizing, a recommended pillow size (for nasal pillow masks), and the like.

In some embodiments, the measurement system 110 may delete the user image(s) 105 and/or mesh 130 after processing in order to preserve user privacy. For example, in some embodiments, once the mesh 130 is generated, the measurement system 110 may delete the images 105 and image data 120. Further, once the measurements 140 are generated, the measurement system 110 may delete the mesh 130. Additionally, in some embodiments, once the interface 150 is generated, the measurement system 110 may delete the measurements 140.

In some embodiments, the measurement system 110 can provide the selected interface 150 to the user depicted in the images 105. In some embodiments, the interface 150 is indicated to another user, such as a healthcare provider of the depicted user, who can facilitate ordering and/or delivery of the indicated equipment. In these ways, the measurement system 110 can use machine learning to generate accurate three-dimensional meshes, and then evaluate these meshes to select or recommend equipment for respiratory therapy in a highly granular way. This can improve the results achieved by users (e.g., improving the progress of the therapy) while reducing or eliminating negative outcomes (e.g., discomfort due to poorly fitted masks, substantial air leak, and the like).

### Example Workflow to Facilitate Image Data Collection for Improved Mesh Generation

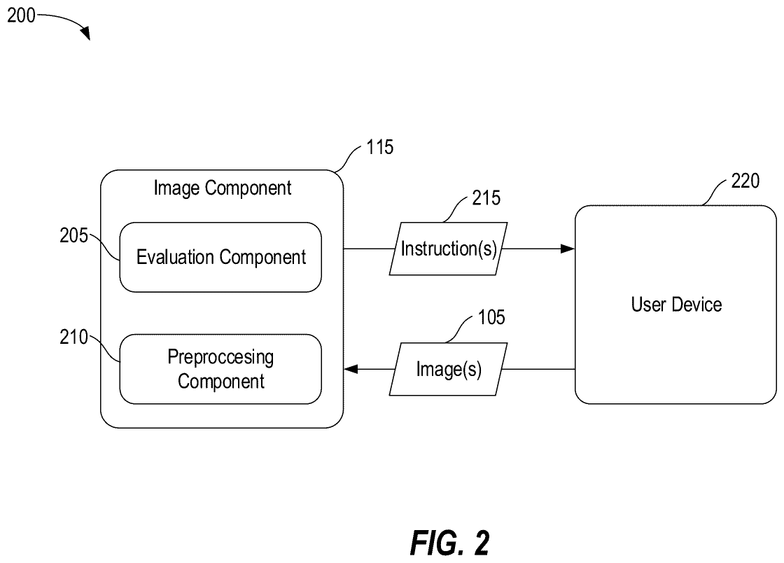

FIG. 2 depicts an example workflow 200 to facilitate image data collection for improved mesh generation, according to some embodiments of the present disclosure. In some embodiments, the workflow 200 may be performed by a measurement system, such as the measurement system 110 of FIG. 1.

As illustrated, the image component 115 of the measurement system comprises an evaluation component 205 and a preprocessing component 210. Although depicted as discrete components for conceptual clarity, in some embodiments, the operations of the depicted components (and others not illustrated) may be combined or distributed across any number of components. In the depicted workflow 200, the image component 115 provides one or more instructions 215 to a user device 220. The instructions 215 may generally include any information to facilitate collection of user images.

For example, the instructions 215 may include textual instructions, pictorial instructions, video instructions, audio instructions, and the like. In some embodiments, the instructions 215 may indicate how the user should position themselves (e.g., by superimposing an ellipse or a human head or face over a live feed from the camera of the user device 220). For example, the instructions 215 may instruct the user to look at the camera, to turn their head to either side of the camera, to look up, and the like. As one example, in some embodiments, the instructions 215 may include depicting or superimposing a box and/or two ellipses (one for each nostril), over the image(s) from the device's camera, and text instructing the user to position their nostrils within the box and/or in the ellipses, and to capture the image when their nostrils are arranged appropriately. Such instructions 215 may enable improved image capture.

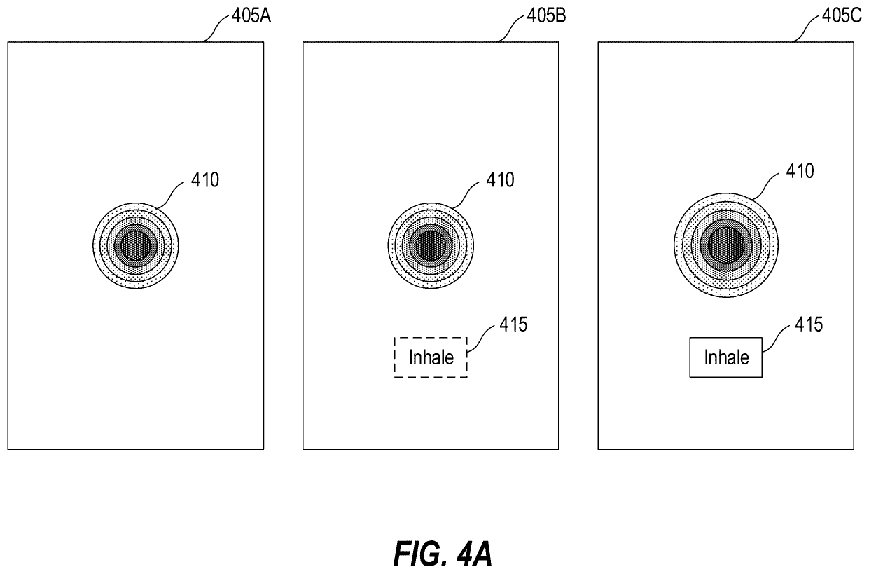

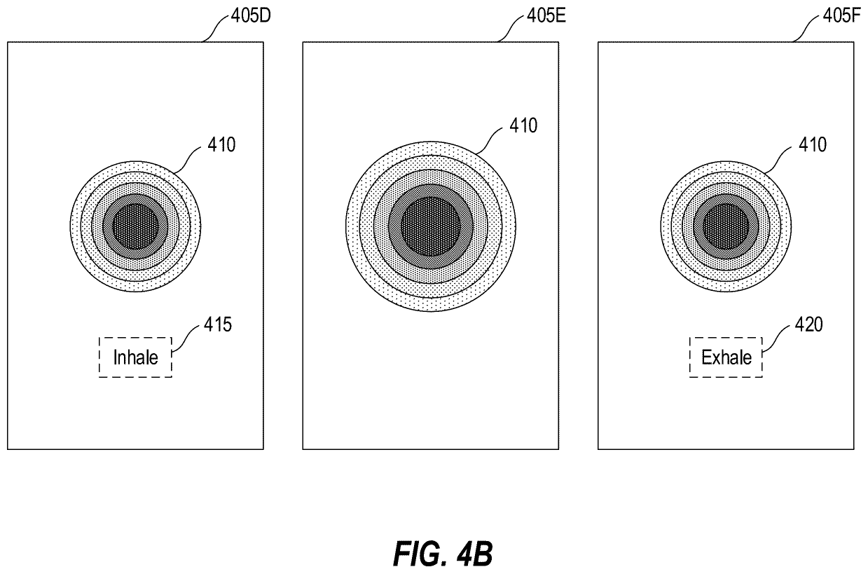

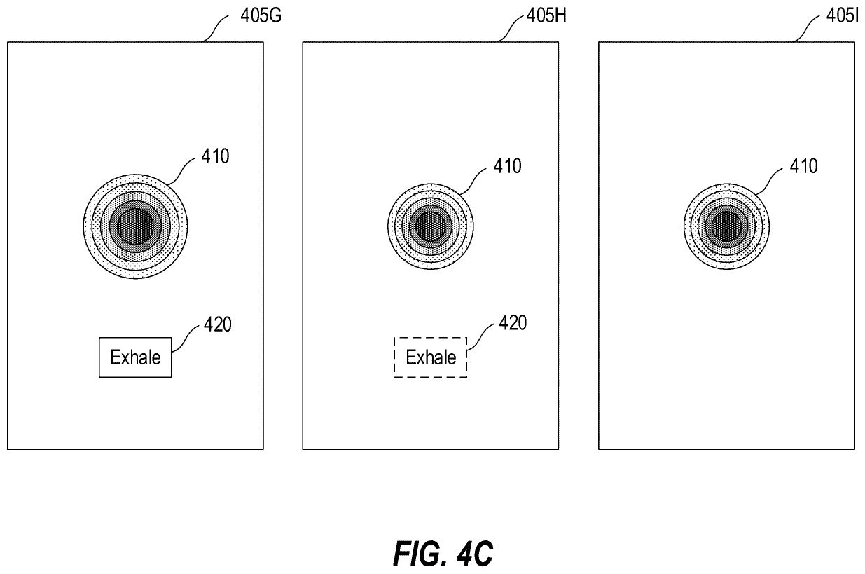

In some aspects, the instructions 215 may include requesting that the user perform a breathing exercise to determine how well the user can breathe through their nose. For example, the instructions 215 may cause the user device 220 to output an animation via a display, and ask the user to breathe (through their nose) in synchronization with the animation. The particular contents of the animation may vary depending on the particular implementation. For example, the animation may include one or more circles (or other shapes) expanding and contracting, asking the user to inhale as the shape(s) expand, and exhale as the shape(s) contract. After one or more such breathing cycles, the instructions 215 may ask the user to indicate whether they were able to breathe comfortably during the exercise (or to rate their level of comfort). One example of such a breathing exercise is discussed in more detail below with reference to FIGS. 4A-C.

Generally, the instructions 215 may be provided using any number and variety of communication links. For example, if the image component 115 operates in a cloud deployment, the instructions 215 may be transmitted via one or more wired and/or wireless networks, (including the Internet) to the user device 220. The user device 220 is generally representative of any computing device that a user may use to capture and/or provide image(s) 105 to the image component 115. For example, the user device 220 may correspond to a laptop computer, a desktop computer, a smartphone, a tablet, and the like. In some embodiments, the user device 220 comprises one or more imaging sensors (e.g., cameras) integrated into the device or available as an external device (e.g., a plugin webcam).

Although not depicted in the illustrated example, in some embodiments, the image component 115 (or another component of the measurement system 110) may provide one or more questions or surveys via the user device 220 to help guide the interface selection process. For example, in some embodiments, the user may be asked whether they have used any other interfaces within a defined period of time (e.g., the last thirty days), and if so, the user may be asked to provide further information such as the model or type of the prior interface(s), the model or type of their current interface, and/or a reason for why they switched (e.g., because they could not get a good seal, because the prior interface was uncomfortable, because they had facial markings or irritation, because they felt claustrophobic with the old interface, because air was leaking and/or they were mouth breathing, because the mask would not stay in place, and the like). In some embodiments, the system may similarly ask the user to indicate whether they initiated the switch (as compared to, for example, their healthcare provider suggesting a switch). Such information may be useful to suggest a new interface for the user (e.g., to select full face, pillow, or nasal mask based on their responses and/or prior interface usage). For example, if the user indicated feelings of claustrophobia while using a full face mask, the measurement system 110 may suggest a nasal or pillow interface.

In some embodiments, the image component 115 (or another component of the measurement system 110) may provide questions related to whether the user breathes through their mouth or otherwise has difficulty breathing through their nose. For example, the user may be asked whether they experience a variety of common concerns (e.g., dry mouth, nasal congestion or irritation, and the like). As another example, the system may ask the user if they have noticed any air leak from their current interface (if they are already participating in therapy), whether they breathe through their mouth when using the therapy, whether they find themselves breathing through their mouth when exerting themselves (e.g., when walking up stairs), whether the user, when asked to take a deep breath, finds it easier to breathe through their mouth or their nose, whether the user has any medical conditions that make breathing through the nose difficult (such as the common cold, chronic sinusitis, chronic allergies, deviated septum, and the like).

In some embodiments, such questions (to determine whether the user tends to breathe through their mouth) may be useful to allow the measurement system to select a good interface recommendation, as discussed above. For example, in addition to recommending specific sizes or models, the measurement system may further recommend specific types based on the user responses (e.g., suggesting a full face mask for users who have difficulty breathing through their nose or who otherwise tend to breathe through their mouth).

As illustrated, the user device 220 transmits one or more image(s) 105 to the image component 115. In the illustrated workflow 200, the preprocessing component 210 may first perform one or more preprocessing operations on the images 105. For example, as discussed above, the preprocessing component 210 may resize the images 105 to a standard or default size, and/or perform a variety of operations such as contrast enhancement and noise reduction to improve the machine learning process.

In the illustrated example, the evaluation component 205 may evaluate the images 105 (or the preprocessed image data generated by the preprocessing component 210) to determine whether the images 105 are acceptable. For example, the evaluation component 205 may use various machine learning models to detect whether the user's face is depicted in the image(s) 105, whether there is sufficient lighting, and the like. In some embodiments, the evaluation component 205 uses a machine learning model trained to identify or detect whether car(s) are depicted in an image 105. For example, the evaluation component 205 may process the image(s) 105 corresponding to when the user turned left and/or right in order to determine whether the user's ear(s) are visible. Such landmarks may be useful to improve the mesh generation, as it may enable more accurate shape and sizing of the model head, which can improve headgear sizing. In some embodiments, if the evaluation component 205 determines that one or more image(s) 105 are not acceptable, the image component 115 can send a new set of instructions 215 to the user asking them to try again (e.g., to take the profile picture again, but move their hair back and out of the way).

In the illustrated workflow 200, this process may be repeated any number of times until an acceptable set of images 105 is obtained.

Although depicted as being performed by the image component 115 of a measurement system, in some aspects, some or all of the discussed operations may be performed locally on the user device 220. For example, the car detection machine learning model may be a lightweight classifier that can be executed by the user device 220 to detect the car visibility locally, allowing the user to immediately capture another image if needed. This may reduce network bandwidth consumed by the process (e.g., reducing the number of images 105 transmitted across the network) as well as reducing computational expense on the measurement system.

Although not depicted in the illustrated workflow 200, in some embodiments, once an acceptable set of images 105 has been generated, the image component 115 may provide the images 105 (or image data generated therefrom, such as feature maps) to one or more other components of the measurement system (e.g., the mesh component 125 of FIG. 1), as discussed above.

### Example Workflow for Improved Interface Selection Based on Generated Meshes

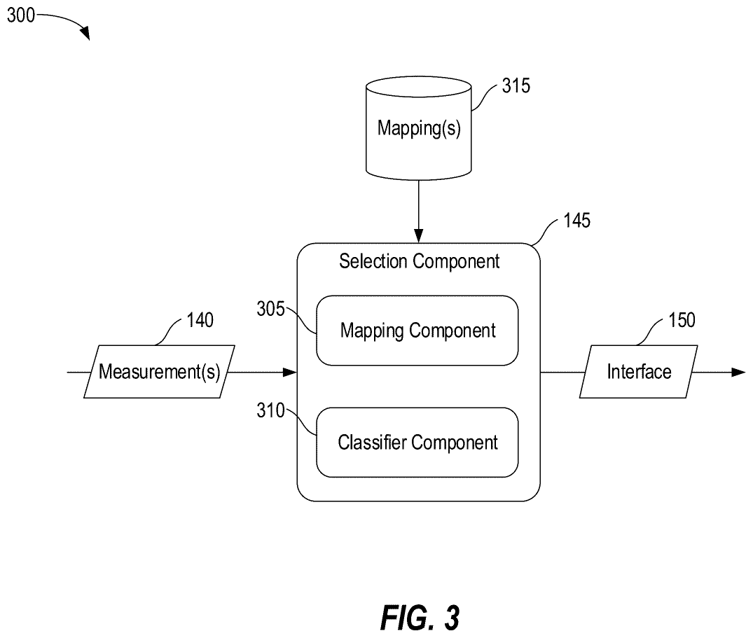

FIG. 3 depicts an example workflow 300 for improved interface selection based on generated meshes, according to some embodiments of the present disclosure. In some embodiments, the workflow 300 may be performed by a measurement system, such as the measurement system 110 of FIG. 1.

As illustrated, the selection component 145 of the measurement system comprises a mapping component 305 and a classifier component 310. Although depicted as discrete components for conceptual clarity, in some embodiments, the operations of the depicted components (and others not illustrated) may be combined or distributed across any number of components. In the depicted workflow 300, the selection component 145 accesses the set of measurements 140 (e.g., facial measurements generated by a measurement component, such as the measurement component 135 of FIG. 1, based on a three-dimensional facial mesh depicting a user, such as the mesh 130 of FIG. 1).

As discussed above, the measurements 140 generally include one or more measurements indicating the size, shape, and/or positioning of one or more facial landmarks in three-dimensional space, such as the relative size, shape, and/or position of the user's eyes, nose, mouth, ears, nostrils, and the like.

In the illustrated example, the selection component 145 also accesses a set of mappings 315. In some embodiments, the mappings 315 generally indicate prior sizing of one or more components of user interfaces. That is, the mappings 315 may indicate, for one or more components (e.g., conduit sizes, interface types or models, headgear sizes, and the like) a range of measurements for which the component was designed and/or which the component will fit appropriately. For example, the mappings 315 may indicate that a first nasal-only mask is best for users having a first set of nose measurements, while a second nasal-only mask is better for users having a second set of nose measurements. In some embodiments, the mappings 315 may be defined or provided by the designers or manufacturers of the therapy components, and/or may be determined based on user interactions (e.g., surveying users to determine which component(s) they prefer).

In some embodiments, the mapping component 305 may evaluate some or all of the measurements 140 using the mappings 315 to select appropriate interface components. For example, as discussed above, the mapping component 305 may select one or more alternatives that align with the measurements 140, such as one or more interfaces, one or more conduits, one or more headgear sizes, and the like.

In the illustrated example, the classifier component 310 may similarly evaluate some or all of the measurements to select appropriate interface components. In some embodiments, as discussed above, the classifier component 310 may process the measurement data using one or more machine learning models in order to select the components. For example, the classifier component 310 may process nostril measurements (e.g., the major and minor axes of an ellipse corresponding to the nostril, the rotation of the nostril, and/or the distance between the nostril and the center of the nose) using a classifier model to select a pillow size for the user.

As illustrated, the selection component 145 generates an interface 150 based on the measurements 140. Although a single interface 150 (e.g., a single set of components) is depicted for conceptual clarity, in some embodiments, the selection component 145 may generate a set of alternatives. For example, the selection component 145 may generate a first interface 150 for a nasal-only style (e.g., recommending a particular interface, conduit, and headgear if the user wants to use a nasal mask), a second interface 150 for a full-face style (e.g., recommending a particular interface, conduit, and headgear if the user wants to use a full-face mask), and/or a third interface 150 for a pillow style (e.g., recommending a particular interface, conduit, headgear, and pillow size if the user wants to use a pillow mask).

Similarly, in some embodiments, the selection component 145 may indicate alternatives within the same style or type of mask. For example, suppose the mappings 315 include overlapping ranges of measurements for one or more components. In some embodiments, if the user's measurements 140 lie in the overlapping region(s), the interface 150 may indicate that any of the alternatives may be suitable.

In these ways, the selection component 145 can provide substantially improved interface selection for users, resulting in improved therapy outcomes and comfort.

### Example Breathing Exercise for Improved User Interface Selection

FIGS. 4A, 4B, and 4C depict an example breathing exercise for improved user interface selection, according to some embodiments of the present disclosure. In some embodiments, the breathing exercise may be used by a measurement system, such as the measurement system 110 of FIG. 1 and/or the measurement systems discussed above with reference to FIGS. 2-3.

The illustrated example depicts a sequence of interfaces 405A-I (e.g., graphical user interfaces (GUIs)), collectively referred to as interface(s) 405 in some aspects, to assist the user in a breathing exercise in order to validate or evaluate the user's ability to breathe through their nose. Although not depicted in the illustrated example, in some aspects, the some or all of the interfaces 405 may depict or include instructions or explanations, such as to explain that the goal of the exercise is to determine how well the user can breathe through their nose, instructions asking the user to inhale and exhale in time (e.g., synchronization) with the animations, and the like. Generally, the instructions or explanations may be provided in a variety of formats, including textual instructions, audio instructions, or a combination of textual and audio instructions.

In some embodiments, the interfaces 405 are output (e.g., displayed) via a user device, such as a smartphone, a laptop computer, a desktop computer, a tablet, and the like. As illustrated in the interface 405A of FIG. 4A, a set of concentric rings or circles 410 are depicted on the interface. In the illustrated example, five concentric circles are depicted. However, there may generally be any number of such circles. Similarly, in some embodiments, any shape may be used to guide the breathing exercise. In the illustrated example, the circles 410 are depicted with a darkest or heaviest shading in the center of the circles 410, progressively getting lighter towards the outer circle. Though the illustrated example depicts a gradient from darker to lighter, in some aspects, the gradient may be reversed. Further, though the illustrated example depicts a discrete set of steps in darkness, in some aspects, the gradient may be a smooth transition (e.g., from dark to light) from the center of the circle to the outer edge. Further, in some aspects, in addition to or instead of using a gradient of color darkness or lightness, the system may instead use a gradient between different colors (e.g., different hues), different saturations, different opacities, and the like.

As illustrated on the interface 405B (which may be displayed subsequent to the interface 405A), a textual instruction 415 (stating “inhale” in the illustrated example) has begun appearing. As indicated by the dashed line around the instruction 415, in some aspects, the instruction 415 may fade in (e.g., from a fully transparent state to a fully opaque state in any color, such as black) over a defined length of time (e.g., selected based at least in part on the desired inhale duration).

As illustrated on the interface 405C, at some point after the interface 405B is output, the instruction 415 reaches full opacity (as indicated by the solid line). Further, as illustrated, the concentric circles 410 have expanded or grown to be a larger size than the size shown on the interfaces 405A and/or 405B. In some aspects, the circles 410 may slowly grow in a smooth or continuous manner (e.g., in synchronization with the text instruction 415 fading in). Although the illustrated example depicts the same set of five circles 410 expanding or growing, in some aspects, the animation may include other visual effects, such as adding or growing additional rings onto the existing circles 410 to expand the total diameter.

Turning to FIG. 4B, on the interface 405D, the instruction 415 is beginning to fade (as indicated by the dashed lines surrounding the instruction 415). For example, this may indicate that the user should be nearing the end of their inhalation (e.g., their lungs should be nearing capacity). Further, as illustrated, the circles 410 have continued to expand, and are now larger than the circles 410 depicted by the interface 405C.

As illustrated by the interface 405E, the textual instruction has fully faded away, and the circles 410 have reached their maximum size (e.g., larger than the circles 410 depicted on the interface 405D). In some aspects, this lack of textual instruction, coupled with the large size of the circles 410, serves to indicate to the user that they should complete their inhalation at this time, with their lungs momentarily full of air.

As illustrated on the interface 405F, after reaching this maximum point (e.g., immediately, or after a brief pause), an additional instruction 420 (stating “exhale” in the illustrated example) begins fading in (e.g., increasing opacity from zero to one hundred percent). Further, the circles 410 on the interface 405F have begun to shrink or contract (e.g., indicating that the user should begin exhaling their breath).

Turning to FIG. 4C, on the interface 405G, at some point after the interface 405F is output, the instruction 420 reaches full opacity (as indicated by the solid line). Further, as illustrated, the concentric circles 410 have continued to shrink or contract to be a smaller size than the size shown on the interface 405F. In some aspects, as discussed above, the circles 410 may slowly contract in a smooth or continuous manner (e.g., in synchronization with the text instruction 420 fading out). Although the illustrated example depicts the same set of five circles 410 contracting, in some aspects, the animation may include other visual effects, such as removing rings from the existing circles 410 to shrink the total diameter.

On the interface 405H, the instruction 420 is beginning to fade back out (as indicated by the dashed lines surrounding the instruction 420). For example, this may indicate that the user should be nearing the end of their exhalation (e.g., their lungs should be nearing empty). Further, as illustrated, the circles 410 have continued to contract, and are now smaller than the circles 410 depicted by the interface 405G.

As illustrated by the interface 405I, the textual instruction has fully faded away, and the circles 410 have reached their minimum size (e.g., the same size as the circles 410 depicted on the interface 405A of FIG. 4A). In some aspects, this lack of textual instruction, coupled with the small size of the circles 410, serves to indicate to the user that they should complete their exhalation at this time, with their lungs momentarily empty.

In some aspects, this sequence can then repeat (e.g., returning to the interface 405A or 405B of FIG. 4A). In some embodiments, by controlling the timing of the transitions between interfaces 405 (e.g., the time that passes while the circles 410 expand from the minimum to maximum size, the time that passes while the inhalation instruction 415 fades in and back out, the time that passes while the circles 410 contract back from their maximum size to their minimum size, and/or the time that passes while the exhalation instruction 420 fades in and back out), the measurement system can guide the speed and depth of the user's breathing for one or more breathing cycles in order to assist the user in determining their ability to breathe through their nose.

For example, if the user finds themselves unable to fully fill their lungs by the time the interface 405E of FIG. 4B is depicted and/or unable to fully empty their lungs by the time the interface 405I of FIG. 4C is depicted, or otherwise begins to feel out of breath during the exercise, the user may realize that they have at least some difficulty breathing through their nose, and may report this difficulty to the measurement system.

In some aspects, as discussed above, the results of this exercise can therefore be used to help select user interface types and/or models. For example, if the user reports no difficulty in completing the exercise (e.g., they were able to fully inhale and exhale, and did not feel that they were receiving inadequate oxygen), the measurement system may determine to suggest a nasal-only and/or pillow user interface, as these interfaces are often believed to be more comfortable for some users.

In contrast, if the user reports difficulty with the exercise, the measurement system may determine potential causes of the difficulty in some embodiments. For example, a follow-up request or question may be provided, such as to ask the user if they are currently suffering from allergies or any other concern that is temporarily impacting their ability to breathe through their nose. If so, the user may still be a good candidate for a nasal-only and/or pillow interface, at least when such maladies are not present. If the user reports no unusual or temporary circumstances causing the difficulty, the measurement system may infer that the user would be better suited with a full-face interface (as compared to a nasal and/or pillow interface). The measurement system may therefore refrain from suggesting a nasal or pillow interface.

In these ways, the dynamic breathing animation exercise can significantly improve the interface selection process, resulting in improved user outcomes.

### Example Method for Using Machine Learning Model(s) to Generate Three-Dimensional Meshes and Select Interface Components

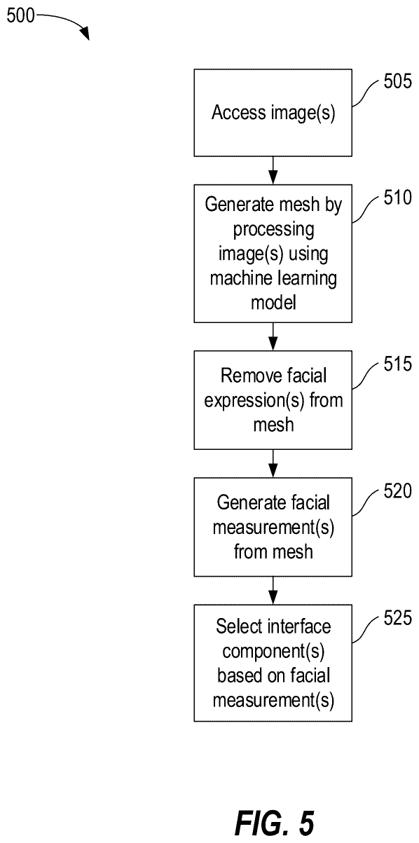

FIG. 5 is a flow diagram depicting a method 500 for using machine learning model(s) to generate three-dimensional meshes and select interface components, according to some embodiments of the present disclosure. In some embodiments, the method 500 may be performed by a measurement system, such as the measurement system 110 of FIG. 1 and/or the measurement systems discussed above with reference to FIGS. 2-4.

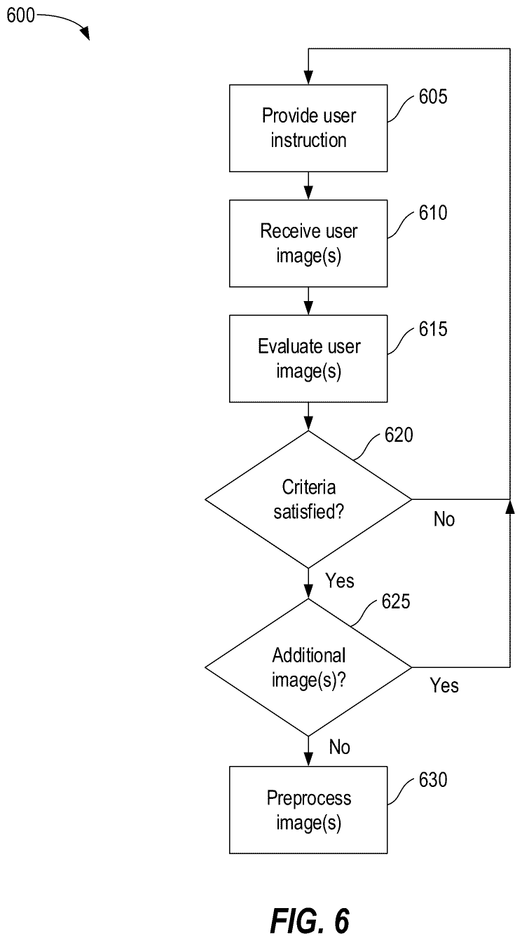

At block 505, the measurement system accesses a set of image(s) (e.g., the images 105 of FIG. 1 and/or FIG. 2). In some embodiments, as discussed above, the images generally depict the head and/or face of a user. In some embodiments, the images are provided in order to enable the measurement system to generate a three-dimensional mesh corresponding to the user's face, allowing the measurement system (or another system) to capture highly accurate measurements relating to the size, shape, positioning, and/or orientation of various facial features or landmarks, such as the nose, nostrils, eyes, mouth, cars, and the like. In some aspects, as discussed above, accessing the images includes evaluating the images to confirm that they meet defined acceptance criteria, such as a minimum size, a minimum resolution, a minimum amount of lighting and/or contrast, and the like. One example method for accessing the images is discussed in more detail below with reference to FIG. 6.

At block 510, the measurement system generates a mesh (e.g., the mesh 130 of FIG. 1) based on processing the accessed image(s) (or image data generated therefrom) using one or more machine learning models (e.g., a deep learning model). For example, as discussed above, the measurement system (or another system) may train a machine learning model using training samples, each sample comprising one or more images (depicting a respective user) as the input and a corresponding set of three-dimensional data points for a set of landmarks on the user's face (or a mesh of the respective user's face) used as the target or label. In some embodiments, as discussed above, some or all of the training samples may comprise synthetic data (e.g., synthetic or artificial face meshes used as the label, with rendered images of the meshes used as the input).

Generally, the particular operations used to train the machine learning model may vary depending on the particular architecture. For example, in some embodiments, the measurement system (or other training system) may process the image(s) of a training sample as input to them model (e.g., a deep learning convolutional neural network) to generate a mesh. The mesh may then be compared against the label (e.g., the actual mesh or other data points in three-dimensional space) to generate a loss. The loss may generally use a variety of formulations, such as surface-to-surface loss, point-to-point loss, surface normal loss, Laplacian regularization loss, and the like. In some embodiments, the parameters of the model may then be updated (e.g., using backpropagation) based on the loss. In this way, the model learns to generate more accurate output meshes based on input images. In embodiments, the model may be trained using individual samples (e.g., using stochastic gradient descent) and/or batches of samples (e.g., using batch gradient descent) over any number of iterations and/or epochs.

In some embodiments, once trained (e.g., once the model reaches a desired level of accuracy, once no additional training samples are available, once a defined number of training iterations or a defined amount of computing resources have been spent, and the like), the model can be used for runtime mesh generation based on input user images.

In some embodiments, as discussed above, the mesh generated by the machine learning model is already scaled to the size of the user's head and/or facial features. That is, because the model may be trained end-to-end using relatively dense labels (e.g., dense point clouds and/or meshes), the model may inherently learn to predict the scale of the face, without separately predicting how the camera affects the perceived size (e.g., based on FOV and/or distance). For example, in some embodiments, the machine learning model learns to respect or recreate the identity (e.g., facial shape) of the person regardless of any variations in angle, background, FOV of the camera, distance of the camera, and the like (in a similar manner to how some facial recognition models work). In this way, the generated mesh may be inherently scaled correctly by the mode.

In some embodiments, if the mesh is not inherently scaled, the measurement system may then scale the output mesh using such a camera model. For example, the measurement system may use the camera model and/or the FOV of the camera (if known) to predict the appropriate size for the mesh (or features therein). For example, objects further from the camera are perceived as smaller, relative to objects nearer to the camera. Therefore, the measurement system may evaluate the change(s) in perceived size of one or more facial landmarks (e.g., the user's ears, nose, mouth, and the like) across the images in order to predict the FOV of the camera, the distance to the landmark(s), and/or the actual size of the feature(s). This allows the measurement system to scale the mesh accurately. In some embodiments, use of a camera model may refer to using a perspective projection technique that projects the mesh from world space to camera space. If the parameters of the camera (e.g., FOV) are known, one or more projected keypoints can be compared with the ground truth keypoint locations (on the image) to determine the appropriate scaling of the mesh.

At block 515, the measurement system removes facial expression(s) present in the mesh, if present. For example, the measurement system may deform the mesh to place the face in a neutral position (e.g., to remove expressions such as smiling, an open mouth, raised eyebrows, and the like). In some embodiments, to remove facial expressions, a statistical model comprising a shape kernel (e.g., indicating a statistically average head shape) and one or more expression kernels (e.g., indicating various facial expressions) may be used. The kernel(s) generally correspond to statistical models generated using principal component analysis (PCA) on facial datasets. For example, for the shape model, the kernel may be generated by performing PCA on a dataset of neutral expressions. For the expression model(s), the kernel(s) may be generated by similarly performing PCA on datasets of various expression(s). In some embodiments, the expression kernel(s) may be used to remove any facial expressions present in the mesh (to cause the mesh to depict or correspond to a neutral expression). As discussed above, removing facial expressions may result in improved measurement accuracy, as compared to taking measurements from a mesh depicting one or more facial expressions. Further, by removing the expression dynamically from the mesh itself, the measurement system may avoid the need to request additional images from the user (e.g., asking the user to take another picture without smiling). This can improve user experience and reduce the time consumed by the measurement process.

At block 520, the measurement system generates one or more facial measurements (e.g., the measurements 140 of FIGS. 1 and/or 3) based on the mesh. For example, as discussed above, the measurement system may generate measurements reflecting features such as the size and shape of the nose, the size and shape of the mouth, the positioning of the mouth relative to the nose, the size, shape, and positioning of the nostrils, the length of the conduit path or other circumferential measure around the user's head (e.g., for the conduit sizing and/or headgear sizing), and the like.

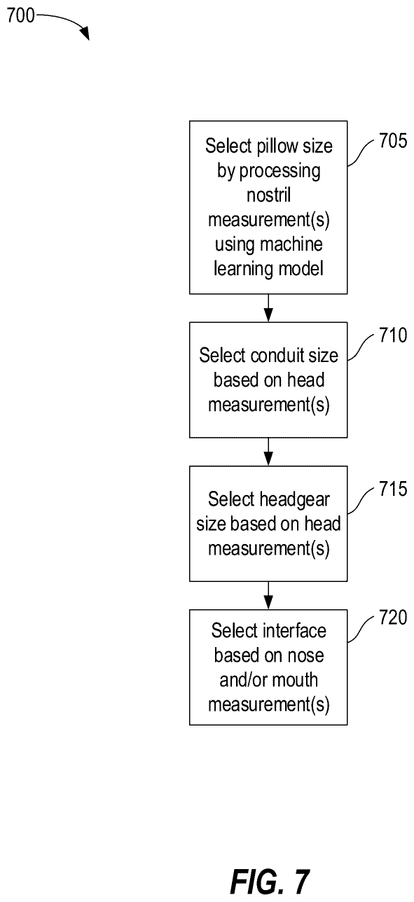

At block 525, the measurement system selects one or more interface components, for the user, based on the facial measurements. For example, as discussed above, the measurement system may select one or more alternatives for each category of component (e.g., one or more conduits, one or more mask types and/or models, and the like) that are well-suited for the user, based on the determined measurements. Although not depicted in the illustrated example, in some embodiments, the measurement system may further select the interface component(s) based at least in part on user responses to survey questions, as discussed above. For example, if the user reports feelings of claustrophobia, the measurement system may select a nasal or pillow style interface. As another example, if the user reports difficulty breathing through their nose and/or tendency to breathe through their mouth, the measurement system may select a full-face style interface. As yet another example, the measurement system may ask the user to engage in a nose breathing exercise (e.g., synchronizing their breathing with an animation), and then ask the user to report how well they could breathe (through their nose) during the exercise. One example breathing exercise is discussed above with reference to FIG. 4. The mask style may be selected based (at least in part) on the user response to this exercise. One example method for selecting the interface components is discussed in more detail below with reference to FIG. 7.

In these ways, using the method 500, the measurement system is able to use machine learning to generate highly accurate three-dimensional meshes based on two-dimensional images, and then collect highly granular facial measurements based on the meshes. This can substantially improve the accuracy of the measurements, resulting in improved reliability in selecting appropriate interface components. As discussed above, these improved selections then enable improved respiratory therapy, such as through increased comfort (which may result in increased uptake or usage of the therapy), decreased air leak or other negative concerns, reduced difficulty or hassle in determining which equipment to select (which may increase the number of patients who decide to start therapy, as the barrier to entry is reduced), and the like. This can substantially improve results for a wide variety of users of respiratory therapy.

### Example Method for Collecting and Evaluating Image Data

FIG. 6 is a flow diagram depicting a method 600 for collecting and evaluating image data, according to some embodiments of the present disclosure. In some embodiments, the method 600 may be performed by a measurement system, such as the measurement system 110 of FIG. 1 and/or the measurement systems discussed above with reference to FIGS. 2-5. In some embodiments, some or all of the method 600 may be performed by other systems, such as locally by the user device (e.g., the user device 220 of FIG. 2) used to capture the images. In some embodiments, the method 600 provides additional detail for block 505 of FIG. 5.

At block 605, the measurement system provides user instructions (e.g., the instructions 215 of FIG. 2) to the user. Generally, as discussed above, the instructions may take a number of forms, such as text instructions, pictorial instructions, video instructions, audio instructions, and the like. The instructions generally indicate what image(s) are needed, such as by illustrating example images that would be acceptable, instructing how the user should angle their head relative to the camera, and the like. In some embodiments, the measurement system may provide instructions for each image independently. For example, the measurement system may provide instructions for a first image in the desired set, capture the image, and then provide instructions for the next image. In other embodiments, the measurement system may provide instructions for all images at once.

In some embodiments, as discussed above, the measurement system may similarly provide one or more questions or surveys to the user (e.g., to infer or determine whether they tend to breathe through their mouth, or to identify any prior interfaces that the user has stopped using). Such information may be useful in providing improved interface selection, as discussed above.

At block 610, the measurement system receives one or more user images (e.g., the images 105 of FIGS. 1-2) from the user device. In some embodiments, as discussed above, the measurement system receives one or more individual images (e.g., the user device may capture one or more images, such as when the user indicates that they are ready). In some embodiments, rather than a set of images, the measurement system receives a video segment (e.g., a stream or sequence of frames). For example, the user device may record a video of the user following the instructions, allowing the measurement system to select the best image(s).

At block 615, the measurement system evaluates the received user image(s) to determine whether the image(s) satisfy one or more defined acceptance criteria. The particular criteria used may vary depending on the particular implementation. For example, in some embodiments, the measurement system may determine whether the image(s) are sufficiently high resolution, have sufficient contrast or clarity, have appropriate lighting, and the like.

In some embodiments, as discussed above, the measurement system may evaluate the image(s) to confirm whether the user followed the instructions appropriately. For example, the measurement system may process the image(s) using one or more computer vision models trained to identify the presence of one or more landmarks or features, such as car(s), eye(s), the mouth, the nose, and the like. As one example for the profile image(s), the measurement system may use an ear detection model to confirm whether the user's ear is visible.

Generally, the particular operations used to train the machine learning models may vary depending on the particular architecture. For example, in some embodiments, the measurement system (or other training system) may process an image of a training sample as input to them model to generate a binary output (or a set of binary outputs) indicating whether one or more landmarks or features are present. The output may then be compared against the label (e.g., whether each landmark is, in fact, present) to generate a loss. The loss may generally use a variety of formulations, such as cross-entropy loss, depending on the particular implementation. In some embodiments, the parameters of the detection model may then be updated (e.g., using backpropagation) based on the loss. In this way, the model learns to predict whether one or more landmarks or facial features are present in provided images. In embodiments, the model may be trained using individual samples (e.g., using stochastic gradient descent) and/or batches of samples (e.g., using batch gradient descent) over any number of iterations and/or epochs.

At block 620, the measurement system determines (based on the evaluation) whether the criteria are satisfied. If not, the method 600 returns to block 605. In some embodiments, the measurement system provides additional instruction at block 605 based on the particular criteria that were not met. For example, the measurement system may specifically indicate that the lighting was poor, that the user was too far from the camera, that the angle was wrong, that the user should ensure their car is visible, and the like.

If, at block 620, the measurement system determines that the criteria are satisfied, the method 600 continues to block 625, where the measurement system determines whether there are one or more additional images, in the desired set of images, which have not yet been provided. If so, the method 600 returns to block 605. If not, the method 600 continues to block 630. Although the illustrated method 600 depicts a sequential process for conceptual clarity (e.g., iteratively receiving and evaluating each image in turn), in some embodiments, the measurement system may receive and/or evaluate some or all of the images in parallel. For example, in some embodiments, the measurement system receives a video of the user moving their head to each designated position (e.g., forward, left, right, up, and down), and may extract appropriate images from this video sequence.

At block 630, the measurement system optionally applies one or more preprocessing operations to the images. As discussed above, the preprocessing operation(s) may generally include any operations to facilitate or improve the machine learning process. For example, the measurement system may adjust the contrast and/or brightness of the images, resize the images, crop the images, and the like. In some embodiments, as discussed above, the measurement system may extract one or more features from the images (e.g., processing the image with a feature extraction machine learning model to generate one or more feature maps), as discussed above.

### Example Method for Improved Interface Selection

FIG. 7 is a flow diagram depicting a method 700 for improved interface selection, according to some embodiments of the present disclosure. In some embodiments, the method 700 may be performed by a measurement system, such as the measurement system 110 of FIG. 1 and/or the measurement systems discussed above with reference to FIGS. 2-6. In some embodiments, the method 700 provides additional detail for block 525 of FIG. 5.

At block 705, the measurement system selects a pillow size for the user by processing one or more nostril measurements using a trained machine learning model. For example, as discussed above, the measurement system (or another system) may train a classifier model to classify nostril measurements into pillow sizes. In some embodiments, as discussed above, the nostril measurements may include parameters such as the major and minor axes of an ellipse that corresponds to the nostril, the rotation of the ellipse or nostril, the distance between the ellipse or nostril and the centerline of the user's nose, and the like. Using such user-specific measurements and machine learning can result in a pillow fitting that is far more comfortable and accurate (as well as far easier and more sanitary, as compared to a guess-and-check approach).

Generally, the particular operations used to train the classifier machine learning model may vary depending on the particular architecture. For example, in some embodiments, the measurement system (or other training system) may process the nostril measurements of a training sample as input to them model to generate a classification (e.g., to select a pillow size). The classification may then be compared against the label (e.g., the actual pillow size appropriate and/or comfortable for the user, based on their nostrils) to generate a loss. The loss may generally use a variety of formulations, such as cross-entropy loss. In some embodiments, the parameters of the model may then be updated (e.g., using backpropagation) based on the loss. In this way, the model learns to generate more accurate pillow size classifications based on input measurements. In embodiments, the model may be trained using individual samples (e.g., using stochastic gradient descent) and/or batches of samples (e.g., using batch gradient descent) over any number of iterations and/or epochs.

At block 710, the measurement system selects a conduit size based on one or more head measurements (e.g., measurements of the length of the conduit path from the user's nose and/or mouth, up across the cheekbones, and over the user's cars). For example, the measurement system may select a dynamic conduit size based on the measurement (e.g., selecting conduit that is the same length as the conduit path), or may select one of a predefined set of alternative conduit sizes (e.g., using a defined mapping between facial measurements and conduit size, such as the mapping 315 of FIG. 3). Using this user-specific measurement can result in a far improved conduit sizing, as compared to more generic or less accurate approaches.

At block 715, the measurement system selects a headgear size based on one or more head measurements (e.g., measurements of the occipitofrontal circumference of the user's head). In some embodiments, to facilitate or improve headgear sizing, the measurement system may fit a statistical shape model of a human head to the mesh, such that the circumference of the head can be estimated (even if the back of the user's head is not imaged). For example, the measurement system may select a dynamic headgear size based on the measurement (e.g., indicating to use headgear that is the same size as the head circumference), or may select one of a predefined set of alternative headgear sizes (e.g., using a defined mapping between facial measurements and headgear size, such as the mapping 315 of FIG. 3). Using this user-specific measurement can result in a far improved headgear sizing, as compared to more generic or less accurate approaches.

At block 720, the measurement system selects a user interface (e.g., a nasal mask, a full-face mask, and/or a nasal pillow mask) based on one or more head or facial measurements (e.g., measurements of the nose and/or mouth of the user). For example, the measurement system may select one of a predefined set of alternative interfaces (e.g., using a defined mapping between facial measurements and interfaces, such as the mapping 315 of FIG. 3). Using these user-specific measurements can result in a far improved interface fit, as compared to more generic or less accurate approaches.

In some embodiments, as discussed above, the measurement system may select multiple interface alternatives. For example, the measurement system may select one interface of each type (e.g., one nasal interface, one nasal pillow interface, and one full-face interfaces), or may select multiple alternatives within each type category. In some embodiments, the particular category (or categories) for which the measurement system generates a selection may depend on user input. For example, the user may specify that they would like a recommended nasal mask. In some embodiments, the measurement system may generate the selection based on predicted user preference or fit (e.g., based on the facial measurements). For example, the measurement system may determine or infer, based on the facial measurements, that a particular type or category of interface will likely be the most comfortable for the user.

In some embodiments, as discussed above, the measurement system may select the interface component(s) based at least in part on user responses to various questions or surveys. For example, the measurement system may select an interface type based on responses related to the user's prior interface usage (e.g., if they already tried a pillow interface and did not like it, for example), based on the user's comfort level with various types, based on the user's tendency to breathe through their mouth or their nose, and the like. As another example, the measurement system may select the interface type based on the user's response to a breathing exercise (e.g., where the user is asked to breathe through their nose in synchronization with an animation), as discussed above with reference to FIG. 4. For example, the measurement system may select a full-face interface type for users who have difficulty breathing through their nose, and a nasal and/or pillow type for users who report potential claustrophobia with full face masks.

Generally, the particular component(s) selected by the measurement system may vary depending on the particular task and implementation. The illustrated examples (e.g., a pillow size, a conduit size, a headgear size, and an interface model) are depicted for conceptual clarity. In various embodiments, however, the measurement system may select additional components not pictured, or may select a subset of the illustrated components, for the user.

### Example Method for Using Machine Learning to Select User Interfaces

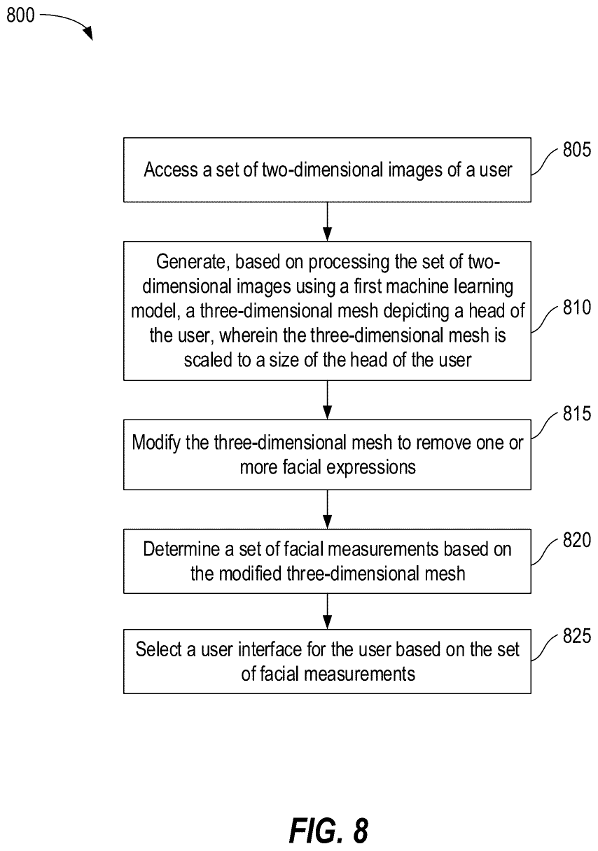

FIG. 8 is a flow diagram depicting a method 800 for using machine learning to select user interfaces, according to some embodiments of the present disclosure. In some embodiments, the method 800 may be performed by a measurement system, such as the measurement system 110 of FIG. 1 and/or the measurement systems discussed above with reference to FIGS. 2-7.

At block 805, a set of two-dimensional images (e.g., the images 105 of FIGS. 1-2) of a user is accessed.

At block 810, a three-dimensional mesh (e.g., the mesh 130 of FIG. 1) depicting a head of the user is generated based on processing the set of two-dimensional images using a first machine learning model, wherein the three-dimensional mesh is scaled to a size of the head of the user.

At block 815, the three-dimensional mesh is modified to remove one or more facial expressions.

At block 820, a set of facial measurements (e.g., the measurements 140 of FIGS. 1 and/or 3) is determined based on the modified three-dimensional mesh.

At block 825, a user interface (e.g., the interface 150 of FIGS. 1 and/or 3) is selected for the user based on the set of facial measurements.

### Example Computing Device for Mesh Generation and Interface Selection

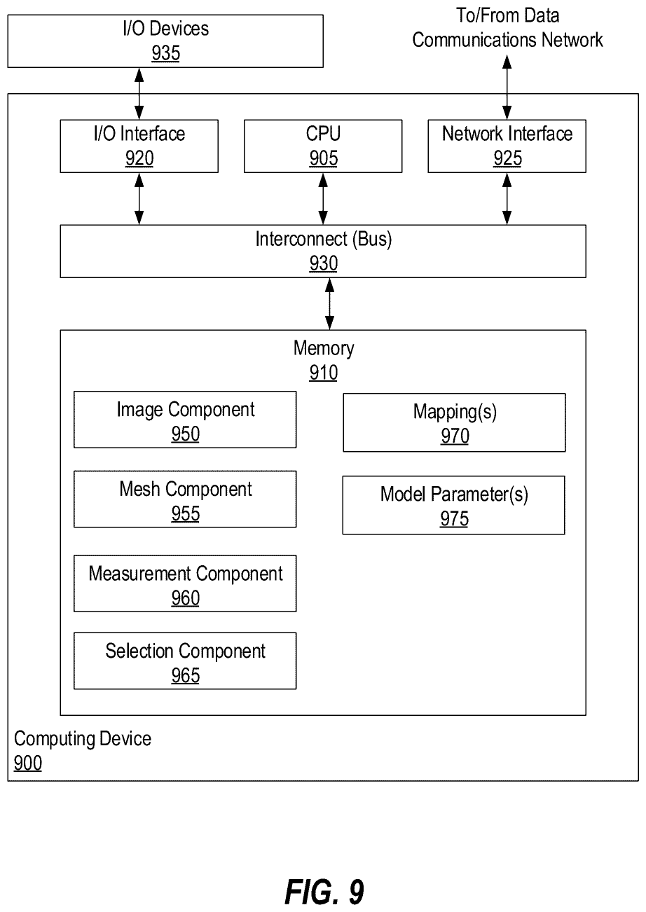

FIG. 9 depicts an example computing device 900 configured to perform various embodiments of the present disclosure. Although depicted as a physical device, in embodiments, the computing device 900 may be implemented using virtual device(s), and/or across a number of devices (e.g., in a cloud environment). In one embodiment, the computing device 900 corresponds to a measurement system, such as the measurement system 110 of FIG. 1 and/or the measurement systems discussed above with reference to FIGS. 2-8.

As illustrated, the computing device 900 includes a CPU 905, memory 910, a network interface 925, and one or more I/O interfaces 920. In the illustrated embodiment, the CPU 905 retrieves and executes programming instructions stored in memory 910, as well as stores and retrieves application data residing in one or more storage repositories (not depicted). The CPU 905 is generally representative of a single CPU and/or GPU, multiple CPUs and/or GPUs, a single CPU and/or GPU having multiple processing cores, and the like. The memory 910 is generally included to be representative of a random access memory. In some embodiments, the computing device 900 may include storage (not depicted) which may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 935 (such as keyboards, monitors, etc.) are connected via the I/O interface(s) 920. Further, via the network interface 925, the computing device 900 can be communicatively coupled with one or more other devices and components (e.g., via a network, which may include the Internet, local network(s), and the like). As illustrated, the CPU 905, memory 910, network interface(s) 925, and I/O interface(s) 920 are communicatively coupled by one or more buses 930.

In the illustrated embodiment, the memory 910 includes an image component 950, a mesh component 955, a measurement component 960, and a selection component 965, which may perform one or more embodiments discussed above. Although depicted as discrete components for conceptual clarity, in embodiments, the operations of the depicted components (and others not illustrated) may be combined or distributed across any number of components. Further, although depicted as software residing in memory 910, in embodiments, the operations of the depicted components (and others not illustrated) may be implemented using hardware, software, or a combination of hardware and software.

In some embodiments, the image component 950 (which may correspond to the image component 115 of FIGS. 1-2) may be used to access, evaluate, and/or preprocess images (e.g., the images 105 of FIGS. 1-2), as discussed above. For example, the image component 950 may transmit or output instructions indicating how to capture the image(s), preprocess the image(s), and/or evaluate the image(s) to confirm that they meet acceptance criteria (e.g., whether an car is visible in the profile image(s)).

In some embodiments, the mesh component 955 (which may correspond to the mesh component 125 of FIG. 1) may be used to generate three-dimensional meshes (e.g., the mesh 130 of FIG. 1) based on two-dimensional images, as discussed above. For example, the mesh component 955 may process the image(s) using one or more deep learning models (or other machine learning models) trained based on image data and corresponding point cloud (or other three-dimensional data, such as mesh data) for user faces.

In some embodiments, the measurement component 960 (which may correspond to the measurement component 135 of FIG. 1) may be used to generate facial measurements (e.g., the measurements 140 of FIGS. 1 and/or 3), as discussed above. For example, the measurement component 960 may collect measurements such defining the size, shape, positioning, and/or rotation of various facial landmarks or features, such as the mouth, the nose, the cars, the nostrils, and the like.

In some embodiments, the selection component 965 (which may correspond to the selection component 145 of FIGS. 1 and/or 3) may be used to generate or select interface components (e.g., the interface 150 of FIGS. 1 and/or 3), as discussed above. For example, the selection component 965 may use mappings (such as the mappings 970) to identify appropriate component(s) based on the facial measurements, and/or may process one or more of the measurements (e.g., the nostril parameters) using one or more secondary machine learning models to predict or identify the best interface component.

In the illustrated example, the memory 910 further includes mapping(s) 970 and model parameter(s) 975 for one or more machine learning models. In some embodiments, the mappings 970 (which may correspond to the mappings 315 of FIG. 3) generally include mappings indicating, for one or more interface components, a set of facial measurements (e.g., a range of measurements) for which the component is acceptable or suitable. Alternatively, the mappings 970 may indicate, for one or more ranges of facial measurements, a set of interface components that are acceptable or suitable. The model parameters 975 may generally include parameters for any number of models, such as a mesh generation model (e.g., a deep learning mode used by the mesh component 955 to generate facial meshes), a landmark or feature detection model (e.g., an car detection model used by the image component 950 to determine whether the profile images are acceptable), a component classifier model (e.g., the nostril pillow classifier model discussed above, used by the selection component 965 to select pillow sizing), and the like.

Although depicted as residing in memory 910 for conceptual clarity, the mappings 970 and model parameters 975 may be stored in any suitable location, including one or more local storage repositories, or in one or more remote systems distinct from the computing device 900.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the embodiments set forth herein. In addition, the scope of the disclosure is intended to cover such an apparatus or method that is practiced using other structure, functionality, or structure and functionality in addition to, or other than, the various embodiments of the disclosure set forth herein. It should be understood that any embodiment of the disclosure disclosed herein may be embodied by one or more elements of a claim.

As used herein, the word “exemplary” means “serving as an example, instance, or illustration.” Any embodiment described herein as “exemplary” is not necessarily to be construed as preferred or advantageous over other embodiments.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., measurement system 110 of FIG. 1) or related data available in the cloud. For example, the measurement system could execute on a computing system in the cloud and train/use machine learning models to generate facial meshes and select interface components. In such a case, the measurement system could maintain the models in the cloud, and use them to drive improved interface recommendations. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

### Example Clauses

Implementation examples are described in the following numbered clauses:
Clause 1: A method, comprising: accessing a set of two-dimensional images of a user; generating, based on processing the set of two-dimensional images using a first machine learning model, a three-dimensional mesh depicting a head of the user, wherein the three-dimensional mesh is scaled to a size of the head of the user; modifying the three-dimensional mesh to remove one or more facial expressions; determining a set of facial measurements based on the modified three-dimensional mesh; and selecting a user interface for the user based on the set of facial measurements.
Clause 2: The method of Clause 1, wherein selecting the user interface comprises generating a recommended pillow size for the user interface based on a set of nostril measurements of the set of facial measurements.
Clause 3: The method of Clause 2, wherein the set of nostril measurements define at least a first ellipse and comprise at least one of: (i) a major axis, (ii) a minor axis, (iii) a rotation, or (iv) a distance of the first ellipse from a center of a nose of the user.
Clause 4: The method of any of Clauses 2-3, wherein generating the recommended pillow size comprises processing the set of nostril measurements using a second machine learning model.
Clause 5: The method of any of Clauses 1-4, wherein selecting the user interface comprises generating a recommended conduit size for the user interface based on the set of facial measurements.
Clause 6: The method of any of Clauses 1-5, wherein selecting the user interface comprises generating a recommended headgear size for the user interface based on the set of facial measurements.
Clause 7: The method of Clause 6, wherein generating the recommended headgear size comprises fitting a statistical shape model of a human head to the three-dimensional mesh.
Clause 8: The method of any of Clauses 1-7, wherein, prior to generating the three-dimensional mesh, at least one two-dimensional image of the set of two-dimensional images was processed using a second machine learning model to detect presence of an ear of the user in the at least one two-dimensional image.
Clause 9: The method of any of Clauses 1-8, wherein the first machine learning model was trained based on a set of training images depicting a training user and a corresponding set of three-dimensional data points for a head of the training user.
Clause 10: The method of Clause 9, wherein the first machine learning model does not use a camera model to generate the three-dimensional mesh.
Clause 11: The method of any of Clauses 1-10, wherein the set of two-dimensional images comprise an image depicting a left side of the head of the user, an image depicting a right side of the head of the user, an image depicting a front of the head of the user, and an image depicting a bottom of the head of the user.
Clause 12: The method of any of Clauses 1-11, further comprising, after selecting the user interface, deleting the set of two-dimensional images, the three-dimensional mesh, and the set of facial measurements.
Clause 13: The method of any of Clauses 1-12, further comprising: providing one or more requests for information to the user, wherein the one or more requests for information ask the user to indicate whether they experience difficulty breathing through their nose receiving, from the user, one or more responses to the one or more requests; and selecting the user interface based further on the one or more responses.
Clause 14: The method of any of Clauses 1-12, further comprising: requesting that the user engage in a breathing exercise by breathing, through a nose of the user, in synchronization with a displayed animation; receiving, from the user, one or more responses to the breathing exercise; and selecting the user interface based further on the one or more responses.
Clause 15: A system, comprising: a memory comprising computer-executable instructions; and one or more processors configured to execute the computer-executable instructions and cause the processing system to perform a method in accordance with any one of Clauses 1-14.
Clause 16: A system, comprising means for performing a method in accordance with any one of Clauses 1-14.
Clause 17: A non-transitory computer-readable medium comprising computer-executable instructions that, when executed by one or more processors of a processing system, cause the processing system to perform a method in accordance with any one of Clauses 1-14.
Clause 18: A computer program product embodied on a computer-readable storage medium comprising code for performing a method in accordance with any one of Clauses 1-14.

What is claimed is:

1. A method, comprising:
accessing a set of two-dimensional images of a user;
generating, based on processing the set of two-dimensional images using a first machine learning model, a three-dimensional mesh depicting a head of the user, wherein the three-dimensional mesh is scaled to a size of the head of the user;
modifying the three-dimensional mesh to remove one or more facial expressions;
determining a set of facial measurements based on the modified three-dimensional mesh; and
selecting a user interface for the user based on the set of facial measurements.

2. The method of claim 1, wherein selecting the user interface comprises generating a recommended pillow size for the user interface based on a set of nostril measurements of the set of facial measurements.

3. The method of claim 2, wherein the set of nostril measurements define at least a first ellipse and comprise at least one of: (i) a major axis, (ii) a minor axis, (iii) a rotation, or (iv) a distance of the first ellipse from a center of a nose of the user.

4. The method of claim 2, wherein generating the recommended pillow size comprises processing the set of nostril measurements using a second machine learning model.

5. The method of claim 1, wherein selecting the user interface comprises generating a recommended conduit size for the user interface based on the set of facial measurements.

6. The method of claim 1, wherein selecting the user interface comprises generating a recommended headgear size for the user interface based on the set of facial measurements.

7. The method of claim 6, wherein generating the recommended headgear size comprises fitting a statistical shape model of a human head to the three-dimensional mesh.

8. The method of claim 7, wherein, prior to generating the three-dimensional mesh, at least one two-dimensional image of the set of two-dimensional images was processed using a second machine learning model to detect presence of an ear of the user in the at least one two-dimensional image.

9. The method of claim 1, wherein the first machine learning model was trained based on a set of training images depicting a training user and a corresponding set of three-dimensional data points for a head of the training user.

10. The method of claim 9, wherein the first machine learning model does not use a camera model to generate the three-dimensional mesh.

11. The method of claim 1, wherein the set of two-dimensional images comprise an image depicting a left side of the head of the user, an image depicting a right side of the head of the user, an image depicting a front of the head of the user, and an image depicting a bottom of the head of the user.

12. The method of claim 1, further comprising, after selecting the user interface, deleting the set of two-dimensional images, the three-dimensional mesh, and the set of facial measurements.

13. The method of claim 1, further comprising:
providing one or more requests for information to the user, wherein the one or more requests for information ask the user to indicate whether they experience difficulty breathing through their nose;
receiving, from the user, one or more responses to the one or more requests; and
selecting the user interface based further on the one or more responses.

14. The method of claim 1, further comprising:
requesting that the user engage in a breathing exercise by breathing, through a nose of the user, in synchronization with a displayed animation;
receiving, from the user, one or more responses to the breathing exercise; and
selecting the user interface based further on the one or more responses.

15. A processing system, comprising:
one or more processors; and
one or more memories collectively comprising computer-executable instructions which, when executed on any combination of the one or more processors, cause the processing system to perform an operation comprising:
accessing a set of two-dimensional images of a user;
generating, based on processing the set of two-dimensional images using a first machine learning model, a three-dimensional mesh depicting a head of the user, wherein the three-dimensional mesh is scaled to a size of the head of the user;
modifying the three-dimensional mesh to remove one or more facial expressions;
determining a set of facial measurements based on the modified three-dimensional mesh; and
selecting a user interface for the user based on the set of facial measurements.

16. The system of claim 15, wherein selecting the user interface comprises generating a recommended pillow size for the user interface based on a set of nostril measurements of the set of facial measurements.

17. The system of claim 16, wherein the set of nostril measurements define at least a first ellipse and comprise at least one of: (i) a major axis, (ii) a minor axis, (iii) a rotation, or (iv) a distance of the first ellipse from a center of a nose of the user.

18. The system of claim 16, wherein generating the recommended pillow size comprises processing the set of nostril measurements using a second machine learning model.

19. The system of claim 15, wherein selecting the user interface comprises generating a recommended conduit size for the user interface based on the set of facial measurements.

20. The system of claim 15, wherein selecting the user interface comprises generating a recommended headgear size for the user interface based on the set of facial measurements.

21. The system of claim 20, wherein generating the recommended headgear size comprises fitting a statistical shape model of a human head to the three-dimensional mesh.

22. The system of claim 21, wherein, prior to generating the three-dimensional mesh, at least one two-dimensional image of the set of two-dimensional images was processed using a second machine learning model to detect presence of an ear of the user in the at least one two-dimensional image.

23. The system of claim 15, wherein the first machine learning model was trained based on a set of training images depicting a training user and a corresponding set of three-dimensional data points for a head of the training user.

24. The system of claim 15, wherein the first machine learning model does not use a camera model to generate the three-dimensional mesh.

25. The system of claim 15, wherein the set of two-dimensional images comprise an image depicting a left side of the head of the user, an image depicting a right side of the head of the user, an image depicting a front of the head of the user, and an image depicting a bottom of the head of the user.

26. The system of claim 15, the operation further comprising, after selecting the user interface, deleting the set of two-dimensional images, the three-dimensional mesh, and the set of facial measurements.

27. One or more non-transitory computer readable media collectively containing, in any combination, computer program code that, when executed by operation of a computing system, performs an operation comprising:
accessing a set of two-dimensional images of a user;
generating, based on processing the set of two-dimensional images using a first machine learning model, a three-dimensional mesh depicting a head of the user, wherein the three-dimensional mesh is scaled to a size of the head of the user;
modifying the three-dimensional mesh to remove one or more facial expressions;
determining a set of facial measurements based on the modified three-dimensional mesh; and
selecting a user interface for the user based on the set of facial measurements.

28. The one or more non-transitory computer readable media of claim 27, wherein selecting the user interface comprises generating a recommended pillow size for the user interface based on a set of nostril measurements of the set of facial measurements.

29. The one or more non-transitory computer readable media of claim 28, wherein the set of nostril measurements define at least a first ellipse and comprise at least one of: (i) a major axis, (ii) a minor axis, (iii) a rotation, or (iv) a distance of the first ellipse from a center of a nose of the user.

30. The one or more non-transitory computer readable media of claim 28, wherein generating the recommended pillow size comprises processing the set of nostril measurements using a second machine learning model.

31. The one or more non-transitory computer readable media of claim 27, wherein selecting the user interface comprises generating a recommended conduit size for the user interface based on the set of facial measurements.

32. The one or more non-transitory computer readable media of claim 27, wherein selecting the user interface comprises generating a recommended headgear size for the user interface based on the set of facial measurements.

33. The one or more non-transitory computer readable media of claim 32, wherein generating the recommended headgear size comprises fitting a statistical shape model of a human head to the three-dimensional mesh.

34. The one or more non-transitory computer readable media of claim 33, wherein, prior to generating the three-dimensional mesh, at least one two-dimensional image of the set of two-dimensional images was processed using a second machine learning model to detect presence of an ear of the user in the at least one two-dimensional image.

35. The one or more non-transitory computer readable media of claim 27, wherein the first machine learning model was trained based on a set of training images depicting a training user and a corresponding set of three-dimensional data points for a head of the training user.

36. The one or more non-transitory computer readable media of claim 27, wherein the first machine learning model does not use a camera model to generate the three-dimensional mesh.

37. The one or more non-transitory computer readable media of claim 27, wherein the set of two-dimensional images comprise an image depicting a left side of the head of the user, an image depicting a right side of the head of the user, an image depicting a front of the head of the user, and an image depicting a bottom of the head of the user.

38. The one or more non-transitory computer readable media of claim 27, the operation further comprising, after selecting the user interface, deleting the set of two-dimensional images, the three-dimensional mesh, and the set of facial measurements.
